(19) United States
(12) Patent Application Publication
(10) Pub. No.: US 2004/0109868 A1
(43) Pub. Date: Jun. 10, 2004

(54) Pim kinase-related methods

(76) Inventors: Paul Rothman, Irvington, NY (US); Peter Chen, New York, NY (US)

Correspondence Address: John P White, Cooper & Dunham, 1185 Avenue of the Americas, New York, NY, 10036, US

(21) Appl. No.: 10/250,380

(22) PCT Filed: Dec. 27, 2001
(86) PCT No.: PCT/US01/50535

(51) Int. Cl. A61K 39/00

(52) U.S. Cl. 424/185.1

(57) ABSTRACT

The present invention provides methods for treating an allergic response, asthma, and the onset of transplant rejection in a subject. These methods involve administering to the subject an agent which increases the amount and/or the activity of a Pim kinase. The present invention also provides a method for determining whether an agent increases the phosphorylation of a Socs-1 protein by a Pim kinase.

This application claims priority of U.S. Provisional Application No. 60/258,421, filed Dec. 27, 2000, the content of which is hereby incorporated into this application by reference.

The invention disclosed herein was made with U.S. government support under grant number RO1 AI3354 from the National Institutes of Health. Accordingly, the U.S. government has certain rights in this invention.

Throughout this application various publications are referenced by Arabic numerals. Full citations for these publications may be found at the end of the specification immediately preceding the claims. The disclosures of these references in their entireties are hereby incorporated by reference into this application in order to more fully describe the state of the art.

### BACKGROUND OF THE INVENTION

One of the mechanisms by which many cytokines exert their effects is through activation of the JAK-STAT signaling pathway. In this pathway, a cytokine initiates signaling by binding to its receptor, thereby inducing receptor oligomerization and activation of the associated nonreceptor JAK kinases. The JAK kinases then phosphorylate specific tyrosine residues within the cytoplasmic domains of the receptor creating docking sites for downstream signaling molecules such as the STAT family of SH2 domain-containing proteins. STAT proteins are phosphorylated by the JAK kinases at the receptor, whereupon they dimerize and translocate to the nucleus. In the nucleus, STAT proteins act as transcription factors that regulate a number of genes involved in hematopoietic cell proliferation and differentiation (reviewed in reference 1)

Several mechanisms have been identified that can control the intensity and duration of JAK-STAT activation. A new protein, SOCS-1/JAB/SSI-1 (for simplicity, referred to as “Socs-1” hereafter), was recently identified as a potent inhibitor of JAK kinase activation (2-4).

Sequence comparison revealed that Socs-1 belongs to a large family of proteins (reviewed in ref. 5). All Socs family members share a conserved C-terminal SOCS box plus either an SH2 or other domain (e.g., WD40 repeats, ankyrin repeats) capable of mediating protein-protein interaction (6). The central SH2 domain of Socs-1 is required for binding to JAK kinases. A sub-domain of approximately 24-amino acids immediately N-terminal to the SH2 domain is critical for maximum inhibition of JAK kinase activity (7, 8).

The levels of Socs-1 appear to be tightly controlled by several mechanisms. Transcription of SOCS-1 mRNA is rapidly induced by many cytokines. Previous work also suggests that SOCS-1 protein stability is regulated. Two groups have reported stabilization of Socs family proteins by inhibitors of the proteasome, suggesting that cells may regulate Socs-1 protein levels through the proteasomal pathway (9, 10). Interestingly, Elongin BC complex, which has been implicated in ubiquitin-mediated degradation, binds to Socs-1 through the SOCS box (10, 11). Association of Elongin BC and the SOCS box has been suggested to alter the stability of the Socs-1 protein.

The Pim serine/threonine kinase family was first identified as a common proviral insertion site in T and B cell lymphomas in mice (12). Three family members have been. identified: Pim-1, Pim-2 and Pim-3. Transcription of the Pim kinases is induced by TCR cross-linking as well as by cytokines such as IL-4, IL-6 and IFN-γ (13-16). Pim-1 and Pim-2 are highly expressed in cells of hematopoietic origin, whereas Pim-3 is undetectable in activated thymocytes and splenocytes (unpublished observation). Forced expression of Pim-1 has been shown to reconstitute thymic cellularity in mice lacking IL-7 or the common gamma chain of cytokine receptors (17). These data suggest that the Pim kinases may play an important role in signaling downstream of cytokine receptors. However, despite extensive investigation, the physiological substrates of the Pim kinases remain unknown.

### SUMMARY OF THE INVENTION

The present invention provides a method for treating an allergic response in a subject which comprises administering to the subject a therapeutically effective amount of an agent which increases the amount and/or the activity of a Pim kinase.

The present invention also provides a method for treating asthma in a subject which comprises administering to the subject a therapeutically effective amount of an agent which increases the amount and/or the activity of a Pim kinase.

The present invention further provides a method for inhibiting the onset of rejection of a transplanted organ, tissue, or cell in a transplant recipient which comprises administering to the transplant recipient a prophylactically effective amount of an agent which increases the amount and/or the activity of a Pim kinase.

In addition, the present invention provides a method for determining whether an agent increases the phosphorylation of a Socs-1 protein by a Pim kinase.

### BRIEF DESCRIPTION OF THE FIGURES

FIG. 1A: Immunoprecipitates of Socs-1 contain Pim-2. 293T cells were co-transfected with plasmids encoding Xpress-tagged Pim-2 and HA tagged SOCS-1. Lysates of transfected cells were immunoprecipitated with either a polyclonal anti-HA antibody (lanes 1 and 3) or normal rabbit serum (lanes 2 and 4). Half of each lysate was then loaded onto separate lanes of an SDS gel and separated by electrophoresis. The presence of the transfected proteins in the immunoprecipitated lysates was detected by standard western immunoblotting techniques using either monoclonal anti-Xpress (lanes 1 and 2) or anti-HA (lanes 3 and 4) antibodies, respectively.

FIG. 1B: Socs-1 is present in Pim-2 immunoprecipitates. 293T cells were co-transfected with plasmids encoding Xpress-tagged SOCS-1 and HA-tagged PIM-2. Lysates were immunoprecipitated with an anti-HA antibody, separated by gel electrophoresis, and analyzed by immunoblotting with either anti-Xpress or anti-HA antibodies as described in FIG. 1A.

FIG. 1C: Association of endogenous Socs-1 and Pim-1. Total thymocytes were isolated from wild-type Balb/c mice, stimulated with PMA and ionomycin for 4 hr and then lysed. Lysates were immunoprecipitated using either pre-immune serum (lanes 1 and 5) or an affinity-purified rabbit anti-Socs-1 antibody (lanes 2 and 6). The immunoprecipitates were analyzed by immunoblotting, first with a goat anti-Socs-1 antibody (C20) (right panel) and then with a monoclonal anti-Pim-1 antibody (left panel). As a control, 50 micrograms of lysate from unstimulated or stimulated thymocytes was loaded in lanes 3 and 4, respectively.

FIG. 1D: The N-terminus of Socs-1 is required for binding to Pim-2. Constructs of Xpress-tagged SOCS-1, full-length (FL) (lanes 1 and 6), ΔN (deleting amino acids 1-79) (lane 2), ΔC (deleting amino acids 167-212) (lane 3), or ΔSH2 (deleting amino acids 80-166) (lane 4) were transiently expressed in 293T cells. Xpress-tagged SOCS-2 was used as a control (lane 5). Whole cell lysates of the transfected 293T cells were incubated with bacterial-expressed Pim-2-GST fusion protein (lanes 1-5) or GST alone (lane 6) bound to GSH agarose beads (Experimental Procedures). Proteins bound to the beads were analyzed by immunoblotting with anti-Xpress antibodies. To ensure equal input, small aliquots of whole cell lysate were subjected to western analysis with anti-Xpress antibodies (lanes 7-12).

FIG. 2A: Co-expression of Socs-1 and Pim-2 results in mobility shift of Socs-1. Plasmids encoding SOCS-1 tagged with Xpress were transfected alone (lanes 1, 4 and 5) or together with PIM-2 (lane 2) or kinase-inactive PIM-2 (K61M) (lane 3) into 293T cells. Equal amounts of a plasmid carrying the LacZ gene were included in each transfection. Whole cell lysates were then analyzed by immunoblotting with anti-Xpress antibodies. 24 hours after transfection, either carrier (DMSO) (lane 4) or 10 micromolar LLnL (lane 5) was added to the cells. Cells were harvested for analysis following an additional 24 hr of culture. The expression levels of wild-type and mutant Pim-2 were comparable (data not shown).

FIG. 2B: The mobility shift of Socs-1 can be reversed by treatment with phosphatase. Lysates of 293T cells transfected with SOCS-1 and Pim-2 were incubated with increasing amounts of X-phosphatase at 30° C. for 90 minutes and analyzed by immunoblotting.

FIG. 2C: N-terminal truncation of Socs-1 abolishes phosphorylation of Socs-1 by Pim-2. Pim-2 and various constructs of Socs-1 were expressed in bacteria as GST fusion proteins. The GST-Pim-2 fusion protein was incubated with full-length (lane 2), N-terminal truncated (lane 3) or C-terminal truncated (lane 4) Socs-1 for in vitro kinase assays (see Methods). GST alone (lane 1) was used as a negative control. A small aliquot of each sample from the kinase assay was analyzed by western blotting with anti-GST antibodies to ensure that the amount of protein in each lane was comparable (lanes 5-8).

FIG. 3A: The phosphorylated form of Socs-1 decays more slowly than the unphosphorylated form. 293T cells were transfected with Xpress-tagged SOCS-1 alone (lanes 1-3) or together with Pim-2 (lanes 4-6). A plasmid carrying the LacZ gene was used as a control. 100 ug/ml of cycloheximide was added to the media 36 hrs after transfection to block new protein synthesis. Cells were harvested at 0, 4.5 and 9 hr time points and total lysates were analyzed by immunoblotting with an anti-Xpress antibody.

FIG. 3B: The blot in (A) was scanned and quantitated using NIH image software, version 1.6.2. The results from three independent experiments are plotted such that the protein level at 0 hr time point is 100 percent.

FIG. 4A: Phosphorylated Socs-1 does not bind Elongin BC as well as unphosphorylated Socs-1 in co-immunoprecipitation experiments. 293T cells were transfected with plasmids carrying Xpress-tagged SOCS-1 alone (lanes 1 and 2) or together with plasmids encoding HA-tagged Elongin B and Elongin C (lanes 3 through 8), in the absence (lanes 3 and 4) or presence of PIM-2 (lanes 5 and 6) or PIM-3 (lanes 7 and 8). Total cell lysates were immunoprecipitated with an anti-HA antibody to pull down Elongin BC. The immunoprecipitates (IP) were then analyzed by Western Blot using an anti-Xpress antibody to detect proteins that bind to Elongin BC (lanes 2, 4, 6 and 8). For comparison, whole cell lysates (WCL) (lanes 1, 3, 5 and 7) were analyzed on the same blot.

FIG. 4B: A GST-Elongin C fusion protein associates preferentially with the faster-migrating band of Socs-1. 293T cells were transfected with plasmids carrying Xpress-tagged SOCS-1 either in the absence (lanes 1 through 3) or presence (lanes 4 through 6) of Pim-1. Total cell lysates from the transfectants were incubated with bacterially expressed GST (lanes 2 and 5) or GST-Elongin C (lanes 3 ad 6) immobilized on glutathione agarose beads. The beads were washed 4 times before being subjected to SDS-PAGE analysis (lanes 3 and 4). Aliquots of whole cell lysates, prior to incubation with glutathione agarose beads were loaded into lanes 1 and 4 as controls.

FIG. 5A: Pim-2 potentiates Socs-1 inhibition of JAK-STAT activation in 293T cells. 293T cells were transfected with 2 ug of p(Iε-IL4RE)4-Luc reporter, 1 ug of pSV4Q-LacZ and 0.6 ug of human Stat6 expression vector by calcium phosphate precipitation. 0.005 ug of plasmid DNA carrying SOCS-1 and 2 ug of plasmid DNA carrying either wild-type PIM-2 or kinase-inactive PIM-2 were used. Total amounts of transfected DNA were kept constant by addition of vector DNA as described (see Methods). Shown on the y-axis is the ratio of luciferase activity between treated and untreated cells. Values reflect means of three independent experiments. The inset shows the expression levels of wild-type Pim-2 and kinase-inactive Pim-2 respectively.

FIG. 5B: Pim-2 potentiates Socs-1 inhibition of JAK-STAT activation in NIH 3T3 cells. NIH 3T3 cells were transfected with 2 ug of p(Iε-IL4RE)4-Luc reporter and 1 ug of pSV40-LacZ as described in Experimental Details. 0.02 ug of plasmid DNA encoding SOCS-1 and 10 ug of PIM-2 plasmid DNA were used. Luciferase assays were performed essentially as described in FIG. 5A.

FIG. 6A: Thymocytes from Pim-1^{-/-}, Pim-2^{-/-} mice and wild-type littermates were stimulated with anti-CD3 (Pharmingen) (1 ug/ml) plus IL-4 (10 ng/ml) and harvested at the indicated time points. Cells were then washed and lysed in 1× NP-40 lysis buffer containing inhibitors of proteases and phosphatases as described (28). Immunoprecipitates were obtained using the anti-Stat6 antibody M20 (Santa Cruz), and blotted with the anti-phospho-tyrosine antibody 4G10 (Upstate Biotechnology).

FIG. 6B: The same blot in FIG. 6A was stripped and re-probed with the anti-Stat6 antibody M20.

FIG. 6C: The levels of endogenous Socs-1 are reduced in the Pim-1^{-/-}, Pim-2^{-/-} mice. Thymocytes were isolated from wild-type (lanes 1 and 2), Pim-¹^{-/-}, Pim-2^{-/-} (lanes 3 and 4) mice and cultured in the presence of PMA and ionomycin for 4 hr. Cells were harvested and lysed, and protein concentration was determined. Equal amounts of lysate were subjected to immunoprecipitation analysis using pre-immune serum (lanes 1 and 3) or an anti-Socs-1 antibody (lanes 2 and 4) as described in FIG. 1C.

### DETAILED DESCRIPTION OF THE INVENTION

Definitions

As used herein, the “activity” of a kinase refers to the phosphoryl group transfer reaction catalyzed thereby.

As used herein, “inhibiting” the onset of a disorder shall mean either lessening the likelihood of the disorder's onset, or preventing the onset of the disorder entirely. In the preferred embodiment, inhibiting the onset of a disorder means preventing its onset entirely.

As used herein “nucleic acid” shall mean any nucleic acid, including, without limitation, DNA, RNA and hybrids thereof. The nucleic acid bases that form nucleic acid molecules can be the bases A, C, G, T and U, as well as derivatives thereof. Derivatives of these bases are well known in the art, and are exemplified in PCR Systems, Reagents and Consumables (Perkin Elmer Catalogue 1996-1997, Roche Molecular Systems, Inc., Branchburg, N.J., USA). Nucleic acids can be “recombinant nucleic acids,” i.e., nucleic acids which do not occur as individual molecules in nature and which are obtained through the use of recombinant DNA technology. Moreover, nucleic acids can exist as vectors which include, for example, plasmid vectors, cosmid sectors and bacteriophage vectors.

The terms “Pim,” “Pim kinase,” “Pim kinase protein” and “Pim protein kinase” are used interchangeably herein. “Pim” shall include, without limitation, Pim-1, Pim-2, Pim-3, and all combinations thereof. “Pim-2” refers to the Pim-2 protein encoded by the gene, PIM-2, as disclosed in reference 30.

As used herein, “polypeptide” shall mean both peptides and proteins. “Peptide” means a polypeptide of fewer than 10 amino acid residues in length, and “protein” means a polypeptide of 10 or more amino acid residues in length. In this invention, the polypeptides may be naturally occurring or recombinant (i.e., produced via recombinant DNA technology), and may contain mutations (e.g., point, insertion and deletion mutations) as well as other covalent modifications (e.g., glycosylation and labeling [via biotin, streptavidin, fluorescein, and radioisotopes]).

As used herein, the term “Socs-1” means the Socs-1 protein that is encoded by the SOCS-1 gene as disclosed in references 2-4, and homologs thereof.

As used herein, “subject” means any animal, including, for example, mice, rats, dogs, guinea pigs, ferrets, rabbits, and primates. In the preferred embodiment, the subject is human.

As used herein, a “transplant recipient” or a “recipient” has the same meaning as a “subject” and refers to any animal, including, for example, mice, rats, dogs, guinea pigs, ferrets, rabbits, and primates. In a preferred embodiment, the transplant recipient is human.

As used herein, “treating” a disorder shall mean slowing, stopping or reversing the disorder's progression. In the preferred embodiment, “treating” a disorder means reversing the disorder's progression, ideally to the point of eliminating the disorder itself.

Embodiments of the Invention

The present invention provides a method for treating an allergic response in a subject which comprises administering a therapeutically effective amount of an agent which increases the amount and/or the activity of a Pim kinase.

The present invention also provides a method for treating an allergic response in a subject which comprises administering to the subject a therapeutically effective amount of an agent which increases the phosphorylation of a Socs-1 protein by a Pim kinase.

The allergic response can be characterized by inflammation. The allergic response can also be characterized by hives, swelling, pain, itching, or redness of skin in the subject.

The present invention also provides a method for treating asthma in a subject which comprises administering a therapeutically effective amount of an agent which increases the amount and/or the activity of a Pim kinase.

The present invention further provides a method for treating asthma in a subject which comprises administering to the subject a therapeutically effective amount of an agent which increases the phosphorylation of a Socs-1 protein by a Pim kinase.

The present invention further provides a method for inhibiting the onset of rejection of a transplanted organ, tissue, or cell in a transplant recipient which comprises administering to the transplant recipient a prophylactically effective amount of an agent which increases the amount and/or the activity of a Pim kinase.

The present invention also provides a method for inhibiting the onset of rejection of a transplanted organ, tissue, or cell in a transplant recipient which comprises administering to the transplant recipient a prophylactically effective amount of an agent which increases the phosphorylation of a Socs-1 protein by a Pim kinase.

The transplanted organ may be a kidney, a heart, an eye, a lung, a stomach, an intestine, an ovary, a pancreas, or at least a portion of liver. The transplanted tissue may be skin, brain, muscle, bone, cartilage, or lung. The transplanted cell may be an islet cell, a bone marrow cell, a blood cell, a bone cell, a cartilage cell, a stem cell, or a plasma cell.

In one embodiment, “increase,” with respect to enzyme activity or amount, means an elevation of at least 1.5-fold thereof, and in another embodiment, means an increase of at least 5-fold.

The agent used in the instant methods can be, for example, a polypeptide, a small molecule or a nucleic acid. Agents contemplated in this invention include, without limitation, (i) a transcription factor which increases the expression of any of the PIM genes encoding members of the Pim family of protein kinases; (ii) an inhibitor of protein phosphatases; (iii) an agonist of a cytokine signaling pathway, (e.g., that of interferon gamma) that induces the expression of any of the PIM family genes; and (iv) a nucleic acid encoding a Pim kinase, operably linked to a promoter.

In the instant methods, cells in which Pim kinase activity is increased include, without limitation, B lymphocytes, T lymphocytes, and mast cells.

Determining a therapeutically or prophylactically effective amount of an agent used in the instant methods can be done based on animal data using routine computational methods. In one embodiment, the therapeutically or prophylactically effective amount contains between about 0.1 mg and about 1 g of the agent. In another embodiment, the effective amount contains between about 1 mg and about 100 mg of the agent. In a further embodiment, the effective amount contains between about 10 mg and about 50 mg of the agent, and preferably about 25 mg thereof.

In this invention, administering agents can be affected or performed using any of the various methods and delivery systems known to those skilled in the art. The administering can be performed, for example, intravenously, orally, via implant, transmucosally, transdermally, intramuscularly, and subcutaneously. In addition, the administered agents ideally contain one or more routinely used pharmaceutically acceptable carriers. Such carriers are well known to those skilled in the art. The following delivery systems, which employ a number of routinely used carriers, are only representative of the many embodiments envisioned for administering the agents.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropyl-methylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropyl-methylcellulose and hyaluronic acid)

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Examples of liposomes which can be used in this invention include the following: (1) CellFectin, 1:1.5 (M/M) liposome formulation of the cationic lipid N,N^{I},N^{II},N^{III}-tetramethyl-N,N^{I},N^{II},N^{III}-tetrapalmityl-spermine and dioleoyl phosphatidylethanolamine (DOPE)(GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-trimethyl-ammonium-methylsulfate) (Boehringer Manheim); and (4) Lipofectamine, 3:1 (M/M) liposome formulation of the polycationic lipid DOSPA and the neutral lipid DOPE (GIBCO BRL).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

This invention further provides the use of each of the agents set forth above for the manufacture of a medicament for performing each of the instant therapeutic and prophylactic methods.

Finally, the present invention provides a method for determining whether an agent increases the phosphorylation of Socs-1 protein by a Pim kinase. This method comprises (i) contacting Socs-1 protein, a Pim kinase, and the agent under conditions which would permit phosphorylation of Socs-1 protein by a Pim kinase in the absence of the agent, (ii) measuring the level of phosphorylation of Socs-1 resulting from step (i), and (iii) comparing that level with the level of phosphorylation of Socs-1 in the absence of the agent, a higher level of phosphorylation in the presence of the agent indicating that the agent increases the phosphorylation of Socs-1 protein by Pim kinase.

In a preferred embodiment, this method further comprises a step of determining whether the increase in phosphorylation of Socs-1 by Pim kinase caused by an agent is specific to that particular kinase reaction. For example, such step can include a control reaction in which either Socs-2 or Socs-3 is contacted with a Pim kinase and the agent under conditions favorable to a kinase reaction. The inability of the agent to alter the phosphorylation of Socs-2 or Socs-3 by Pim kinase would indicate that the agent specifically enhances the reaction between Socs-1 and Pim kinase.

This invention is illustrated in the Experimental Details section which follows. This section is set forth to aid in an understanding of the invention but is not intended to, and should not be construed to, limit in any way the invention as set forth in the claims which follow thereafter.

### EXPERIMENTAL DETAILS

Synopsis

Studies of SOCS-1 deficient mice have implicated Socs-1 in the suppression of JAK-STAT signaling and T cell development. It has been suggested that the levels of Socs-1 protein may be regulated through the proteasome pathway. Here we show that Socs-1 interacts with members of the Pim family of serine/threonine kinases in thymocytes. Co-expression of the Pim kinases with Socs-1 results in phosphorylation and stabilization of the Socs-1protein. The protein levels of Socs-1 are significantly reduced in the Pim-1^{-/-}, Pim-2^{-/-} mice as compared to wild-type mice. Similar to Socs-1 null mice, thymocytes from Pim-1^{-/-}, Pim-2^{-/-} mice showed prolonged Stat6 phosphorylation upon IL-4 stimulation. These data suggest that the Pim kinases may regulate cytokine induced JAK-STAT signaling through modulation of SOCS-1 protein levels.

Methods

Yeast Two-Hybrid Screen

Full-length murine SOCS-1 was subcloned into the pAS2 vector (Clonetech, USA) , and transformed into the yeast strain Y190. A murine macrophage cDNA library employing the PGADNOT vector was used. Yeast transformation was carried out using the EZ Yeast Transformation kit from Zymo Research (Orange, Calif.) and yeast two-hybrid screen was conducted essentially as described by Durfee et al. (18).

Generation of Socs-1 Antibodies

1.5 mg of recombinant GST-Socs-1 fusion protein was expressed and purified from bacteria and injected into a rabbit for production of anti-Socs-1 serum. The third bleed which had the highest titer was incubated overnight with Glutathione beads (Sigma) cross-linked to GST to eliminate antibodies specific for GST. The pre-cleared serum was then loaded at a flow rate of 10 ml/hr onto an affinity column packed with Glutathione beads cross-linked to GST-Socs-1 fusion protein. The column was washed at a rate of 1 ml/min with 10 times the column volume of 1× TBS (20 mM Tris-HCl, pH 7.5, 150 mM NaCl) and then with 10 times the column volume of 0.1× TBS. After the last wash, Socs-1 specific antibodies were eluted with one column volume of 0.1M Glycine-HCl, pH 2.5, into tubes pre-loaded with equal volume of 1 M Tris base.

Biochemical Experiments

Murine SOCS-1 was subcloned into a mammalian expression vector pcDNA3.1 HIS A (Invitrogen) with an in-frame Xpress tag. Murine Pim-2 was subcloned into another mammalian expression vector pCGN with an inframe HA tag. Anti-Xpress antibodies were from Invitrogen, rabbit polyclonal anti-HA antibodies and normal rabbit serum were from Santa Cruz Biotechnology, Inc. Co-immunoprecipitation experiments were carried out as previously described (28). LLnL (N-acetyl-leu-leu-norleucinal) and cycloheximide were purchased from Sigma. Gamma-phosphatase was from New England Biolabs. To detect endogenous Socs-1 protein, thymocytes were removed from 10 wild-type Balb/c mice (4 wk-old). Total thymocytes were cultured in the presence of 50 ng/ml phorbol myristate acetate (PMA, Sigma) and 500 ng/ml ionomycin (Sigma) for 4 hours. Cells were lysed in buffer containing 1% NP-40, 50 mM Tris, pH 8.0, 2 mM EDTA, 5 ug/ml each of the protease inhibitors: leupeptin, aprotinin and pepstatin, 1 mM PMSF, 1 mM sodium orthovanadate and 50 mM NaF. Cell lysates were subjected to immunoprecipitation using normal rabbit serum or affinity-purified rabbit anti-Socs-1 antibodies. The immunoprecipitates were loaded onto a 12% SDS-polyacrylamide gel and immunoblotted with goat anti-Socs-1 antibody (C20, Santa Cruz)

GST Pull-Down Experiments

Pim-2 was subcloned into the pGEX vector (Pharmacia). GST fusion proteins were expressed in the bacteria DH5alpha. The conditions for expression of GST fusion proteins and for the GST pull-down experiments are as described in (29). Socs-1 ΔN, Socs-1 ΔC and Socs-1 ΔSH2 mutants were generated by PCR followed by subcloning into the pcDNA3.1 vector.

In Vitro Kinase Assay

Various SOCS-1 mutants were subcloned into the pGEX vector (Pharmacia). Expression of GST fusion proteins was as described (29), except for the GST-Socs-1 fusion proteins, which were induced at 30° C. GST fusion proteins of Pim-2 and Socs-1 were incubated together with ³²P-γ-ATP essentially as described (30). The reactions were washed three times and fractionated by SDS-PAGE. The gel was subjected to drying and autoradiography. A small aliquot of each reaction was analyzed by Western blotting using an anti-GST antibody.

Transient Luciferase Assay

Luciferase assays were performed as described (31) with some modifications: 293T cells were transfected by the calcium phosphate precipitation method. 24 hours after transfection, cells of each transfection were divided in half. One half was treated with human IL-4, the other half served as a control. Cells were harvested 18 hr after treatment. Half of the cells were processed to determine luciferase activity, while the other half was subjected to Western blot to compare the expression levels of Pim-2 protein. NIH 3T3 cells were transfected using the Lipofect amine reagent from Life Technologies (Maryland, USA) according to the manufacturer's protocol.

Results

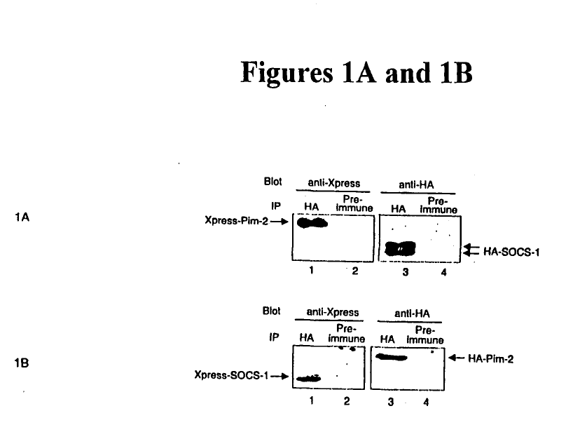

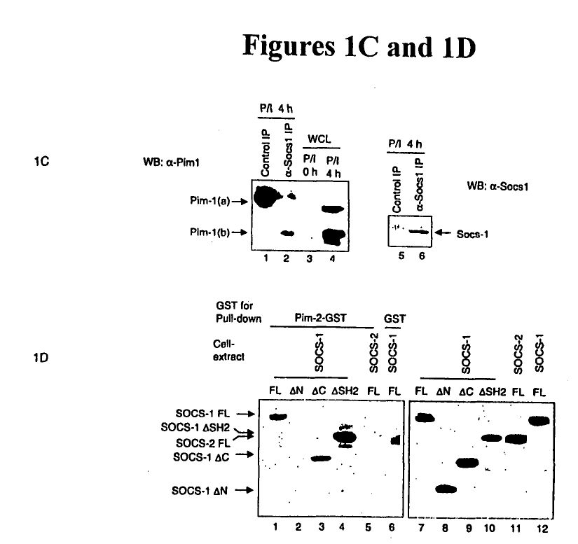

To identify proteins that interact with Socs-1, a yeast two-hybrid screen was conducted using full-length murine SOCS-1 as bait. A murine cDNA library from macrophages was screened as described in Durfee et al. (18). One of the genes identified encodes the serine/threonine kinase PIM-2. To confirm the interaction between Socs-1 and Pim-2 in mammalian cells, plasmids encoding epitope-tagged PIM-2 and SOCS-1 were transfected into 293T cells. Cell lysates were subjected to co-immunoprecipitation analysis. Pim-2 was detected in Socs-1 immunoprecipitates by immunoblot analysis (FIG. 1A). Conversely, Socs-1 was detected in the Pim-2 immunoprecipitates (FIG. 1B). In addition to Pim-2, Socs-1 also interacted with Pim-1 and Pim-3 in co-immunoprecipitation experiments (data not shown). To determine if Socs-1 can bind to Pim kinases under physiologic conditions, thymocytes were isolated from wild-type mice and stimulated with PMA and ionomycin for 4 hr before being subjected to lysis and immunoprecipitation (12). As expected, two isoforms of Pim-1 (Pim-1 (a) and Pim-1 (b)) were induced by PMA and ionomycin treatment (FIG. 1C, lanes 3 and 4). Interestingly, the smaller form Pim-1 (b) was associated with endogenous Socs-1 more strongly than the larger form Pim-1 (a) (FIG. 1C, lane 2), since co-immunoprecipitation of Pim-1 (a) was only detectable when the same blot was exposed much longer (data not shown). Thus, Socs-1 protein interacts with the Pim family of kinases in vivo.

To map the domain of Socs-1 that mediates its interaction with Pim-2,-lysates of 293T cells transfected with various truncation mutants of Socs-1 were incubated with Pim-2-GST (glutathione-S-transferase) fusion protein immobilized on agarose beads. Socs-1 lacking either the SH2 domain (ΔSH2) or the C-terminal SOCS box (ΔC) could bind to Pim-2 as well as full-length (FL) Socs-1. In contrast, truncation of the N-terminal 79 amino acids of Socs-1 (ΔN) abolished its association with Pim-2 (FIG. 1D) As a control, Socs-2, another member of the SOCS family, did not bind to Pim-2. Taken together, these results indicate that Pim-2 specifically binds to Socs-1, and that this interaction is mediated by the N-terminus of Socs-1.

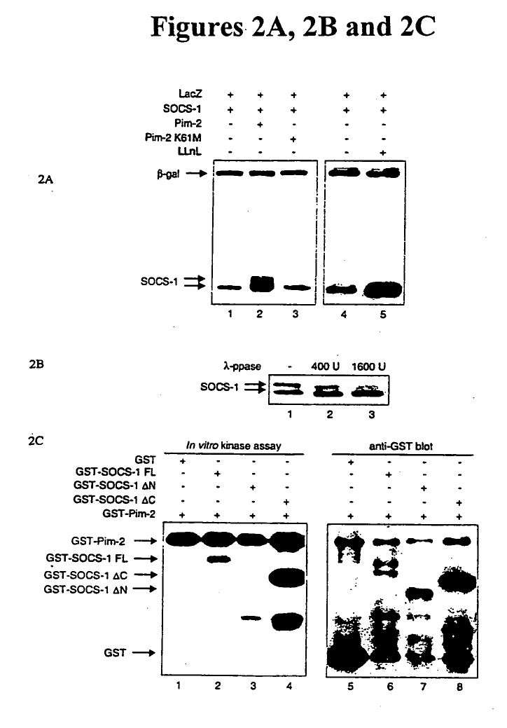

Strikingly, when Socs-1 and Pim-2 were co-expressed in 293T cells, a slower-migrating Socs-1 isoform was observed (FIG. 2A). Co-expression of Socs-1 with kinase-inactive Pim-2 did not result in the slower-migrating Socs-1 band, even though the expression levels of wild-type and mutant Pim-2 were comparable (data not shown). Pim-1 or Pim-3 also caused a mobility shift of Socs-1 when they were co-expressed in 293T cells (unpublished results). In contrast, no mobility shift was observed when either Socs-2 or Socs-3 was co-expressed with the Pim kinases (data not shown), implying that Socs-1 may be a specific substrate for Pim kinases. To determine whether the slower-migrating band is a phosphorylated form of Socs-1, total cell lysates of 293T cells expressing Socs-1 and Pim-2 were incubated with λ-phosphatase. Upon phosphatase treatment, the intensity of the slower-migrating Socs-1 band decreased, while the levels of the faster-migrating band increased (FIG. 2B). Thus, Pim kinases are capable of phosphorylating Socs-1 in vivo, and this modification can be reversed by phosphatase treatment.

As the slower-migrating Socs-1 species is likely the result of phosphorylation by serine/threonine kinases, we sought to determine if Socs-1 is a direct substrate of the Pim-2 kinase. An in vitro kinase assay was performed using GST-Pim-2 and GST-Socs-1 fusion proteins. While Pim-2 did not phosphorylate the GST protein, it phosphorylated the full-length Socs-1-GST fusion protein in vitro. The C-terminal SOCS box is dispensable for phosphorylation as a Socs-1 mutant lacking the SOCS box (ΔC) was still phosphorylated (FIG. 2C). Strikingly, deletion of the N-terminal 79 amino acids of Socs-1 (ΔN) abolished its phosphorylation by Pim-2 completely (FIG. 2C, lane 3). Although the N-terminus of Socs-1 is required for its interaction with Pim-2, the GST moiety, through GST dimerization, was presumably able to mediate an interaction between Pim-2 and N-terminal truncated Socs-1. Therefore, abrogation of phosphorylation is likely not due to a lack of interaction between Socs-1 and Pim-2, rather, it is most likely the result of the loss of phosphorylation sites at the N-terminus of Socs-1. Consistent with the in vitro kinase assay, Pim-2 failed to cause a mobility shift of the Socs-1 ΔN mutant when they were co-expressed in 293T cells (unpublished observation) These results suggest that Socs-1 is a direct substrate for the Pim-2 kinase and that, at least in vitro, the major phosphorylation sites are within the N-terminal 79 amino acids of Socs-1.

Interestingly, co-expression of Pim-2 and Socs-1 in 293T cells drastically increased the steady-state levels of Socs-1, an effect which resembles the stabilization of Socs-1 protein by the proteasomal inhibitor LLnL (FIG. 2A, lanes 1, 2, 4 and 5). The kinase activity of Pim-2 was required for this stabilization effect, since a kinase-inactive mutant of Pim-2 failed to increase the protein levels of Socs-1 (FIG. 2A). As a control for transfection efficiency, a plasmid carrying the LacZ gene was included in each transfection. The levels of 3-galactosidase encoded by the LacZ gene did not change while the protein levels of Socs-1 increased in the presence of Pim-2 or LLnL.

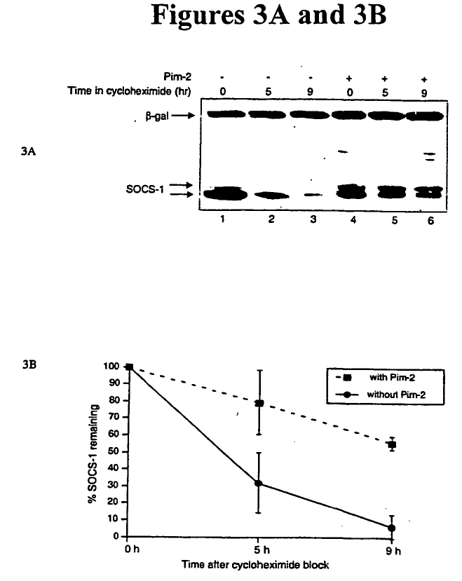

The increase in Socs-1 protein levels in the presence of the Pim kinases can be attributed to augmented production or decreased degradation of the Socs-1 protein. In order to distinguish between these two possibilities, the levels of Socs-1 were monitored following addition of cycloheximide to block protein synthesis. Socs-1 was transiently transfected into 293T cells in the absence or presence of Pim-2. Thirty-six hours after transfection, cycloheximide was added to the culture to block further protein synthesis, and the decay of the Socs-1 protein was measured by immunoblotting. In the absence of Pim-2, less than 10% of the Socs-1 protein remained after 9 hours in cycloheximide. In contrast, when Pim-2 was present, more than 60% of Socs-1 remained after the same period of time (FIGS. 3A and 3B). As a control for transfection efficiency and protein loading, the levels of β-galactosidase encoded by a co-transfected plasmid remained steady. Interestingly, the slower-migrating band of Socs-1 persisted longer than the band with faster mobility (FIG. 3A, lanes 4, 5 and 6), indicating that phosphorylation renders Socs-1 more stable. Consistent with the cycloheximide experiment, the half-life of Socs-1 protein was prolonged by co-expression of the Pim kinases in a pulse-chase experiment (data not shown). Moreover, the stabilization of Socs-1 by Pim kinases appears to be specific, as the decay of Socs-3 was not significantly affected by co-expression of the Pim kinases under the same conditions (data not shown). Together, these data suggest that phosphorylation by the Pim kinases down-regulates degradation of Socs-1 protein, thus augmenting the levels of Socs-1 protein in the cells.

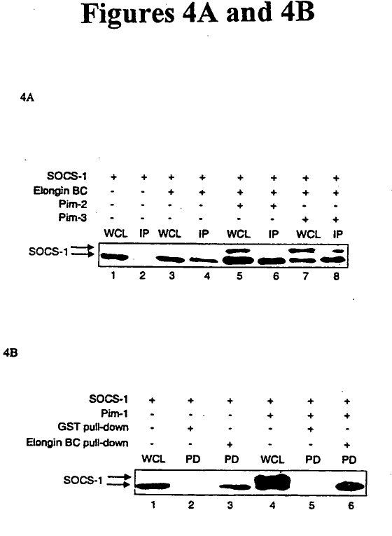

The stability of the Socs-1 protein has been reported to be altered by its association with Elongin BC (10, 11). We thus sought to determine if the Pim kinases have any effect on the association of Socs-1 and Elongin BC. Socs-1 was co-expressed in 293T cells with Elongin BC in the presence or absence of Pim-2 or Pim-3. The formation of the Socs-1 doublet was not altered by co-expression of Elongin BC (FIG. 4A, lanes 1, 3, 5 and 7). Interestingly, when the cell lysates were subjected to immunoprecipitation using anti-Elongin C antibodies, the fast-migrating band of Socs-1 preferentially co-immunoprecipitated with Elongin BC (FIG. 4A, lanes 6 and 8). To confirm this result, a GST pull-down experiment was also conducted. When bacterial-expressed GST-Elongin C fusion protein was incubated with lysates of 293T cells transfected with SOCS-1 alone or together with the Pim kinases, GST-Elongin C associated specifically with the fast-migrating band of Socs-1 (FIG. 4B, lanes 4 and 6). As the slow-migrating band likely represents a phosphorylated form of Socs-1, these results suggest that phosphorylation of Socs-1 by the Pim kinases decreases the interaction between Socs-1 and Elongin BC.

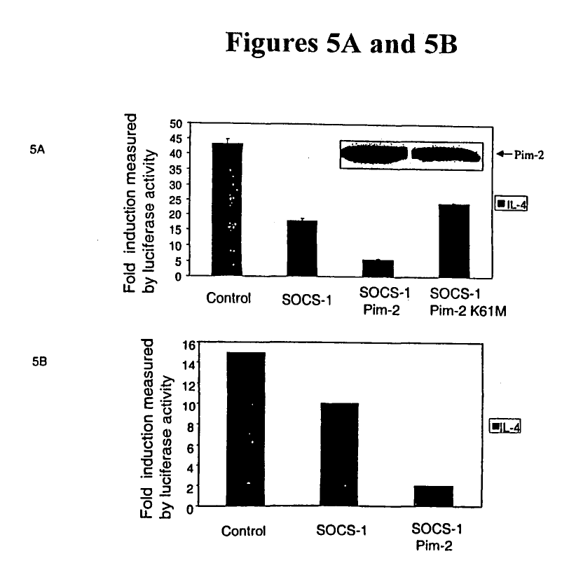

In order to determine the functional significance of the interaction between the Pim kinases and Socs-1, the effect of Pim-2 on IL-4 mediated Stat6 activation was evaluated by transient luciferase assays. We have previously reported that Socs-1 can inhibit IL-4 induced Stat6 activation in 293T cells (19). Co-transfection of Pim-2 with SOCS-1 further inhibited Stat-mediated reporter expression, while kinase inactive Pim-2 had no effect (FIG. 5A). The same effect by Pim-2 was observed in NIH 3T3 cells (FIG. 5B).

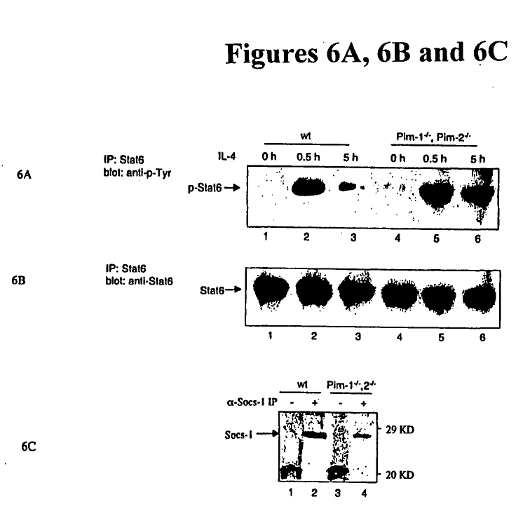

To elucidate the biochemical mechanisms by which Pim kinases affect cytokine signaling, primary thymocytes from Pim-1^{-/-}, Pim-2^{-/-} mice or wild-type littermates were treated with IL-4, and the status of Stat6 tyrosine phosphorylation was assessed. Five hours after IL-4 stimulation, tyrosine phosphorylation of Stat6 was almost completely abrogated in wild-type cells. In contrast, significant amounts of Stat6 remained phosphorylated in the Pim-1^{-/-}, Pim-2^{-/-} cells after the same period of time (FIGS. 6A and 6B).

Interestingly, thymocytes from SOCS-1 null mice also exhibit prolonged activation of Stat6 after IL-4 treatment (20). This defect in down-regulating JAK-STAT signaling may be due the lack of adequate amounts of Socs-1 when both Pim-1 and Pim-2 are absent. We thus examined the levels of Socs-1 protein in these mice. Thymocytes from either wild-type or Pim-1^{-/-}, Pim-2^{-/-} mice were treated with PMA and ionomycin for 4 hours. Cells were lysed and subjected to anti-Socs-1 immunoprecipitation and immunoblot analysis. The levels of Socs-1 protein were significantly higher in wild-type than in the mutant mice (FIG. 6C). As a control, anti-Lck immunoprecipitation was carried out using the same lysates, and no significant difference of Lck levels were observed (data not shown). Taken together, these findings suggest that the Pim kinases help maintain the levels of Socs-1 protein and thus potentiate Socs-1 inhibition of JAK-STAT activation.

Discussion

SOCS-1 was first identified as an auto-feedback inhibitor of JAK kinases. The current model is that cytokine stimulation activates JAK-STAT signaling, which in turn triggers the transcription of SOCS-1. The resultant Socs-1 protein translocates to the cytokine receptors and suppresses the kinase activity of JAK. As a potent inhibitor of JAK kinases, the levels of Socs-1 must be tightly regulated. Here we show that Socs-1 is a labile protein and that its degradation is regulated by Pim-mediated serine/threonine phosphorylation.

In thymocytes, the steady-state levels of Socs-1 are increased by LLnL (unpublished observation), suggesting that the levels of Socs-1 protein may be regulated through the proteasome pathway. Although the exact mechanism by which the Socs-1 protein is degraded is not well understood, previous studies on the interaction between SOCS-1 and Elongin BC have suggested a role for Elongin BC in the degradation of Socs-1 (10, 11). However, the precise role of Elongin BC remains controversial. While Kamura et al (11) have provided evidence that Elongin BC complex helps stabilize Socs-1, Zhang et al (10) have suggested that Elongin BC complex targets Socs-1 to the proteasomal degradation pathway. Our data demonstrate that phosphorylation of Socs-1 by the Pim kinases decreases the binding between Socs-1 and Elongin BC and down-regulates Socs-1 degradation. Moreover, in preliminary experiments, a Socs-1 mutant (L175P, C179F) that fails to bind to Elongin BC is more stable than wild-type Socs-1 (unpublished observation), suggesting that Elongin BC complex negatively affects the stability of Socs-1 protein.

Socs-1 mRNA is expressed at the highest level in the thymus, and SOCS-1 deficient mice manifest defects in cytokine signaling as well as in T-cell development (20-22). Interestingly, both Pim-1 and Pim-2 have been implicated in thymocyte development (17, 23). Forced expression of Pim-1 has been shown to rescue the defects in thymocyte development caused by deficiency in RAG, IL-7 or the common gamma chain of cytokine receptors (17). Moreover, while Pim kinases have been shown to be up-regulated by cross-linking of T cell receptors, Socs-1 transcription does not seem to be induced by TCR signaling (22). However, when thymocytes were activated with PMA and ionomycin or anti-CD3, which mimis cross-linking of the T cell receptor, the protein levels of both Socs-1 and Pim-1 were augmented (unpublished observation). Northern blot analysis indicated that the levels of Socs-1 mRNA were unchanged, suggesting that the levels of Socs-1 may be post-transcriptionally regulated. The hypothesis that Pim kinases stabilize Socs-1 post-transcriptionally was further strengthened by the observation that the protein levels of Socs-1 are much lower in the Pim-1^{-/-}, Pim-2^{-/-} mice than in wild-type mice.

Interestingly, while we consistently observe a mobility shift of the socs-1 protein when it is co-expressed with the Pim kinases, endogenous Socs-1 is not detected as a doublet even when Pim kinases are abundant. It is possible that the single band of endogenous Socs-1 is phosphorylated and mobility-shifted, since it migrates slower than would be predicted from the calculated molecular weight. The unphosphorylated form of endogenous Socs-1 is not detectable because it may be rapidly degraded under physiologic conditions. Alternatively, endogenous Socs-1 maybe phosphorylated by Pim kinases to an extent that does not result in such a dramatic mobility shift as observed in the over-expression system.

The Pim kinases and Socs-1 are all induced by a variety of cytokines and the interaction between Pim and Socs-1 appears to represent a novel mechanism by which cytokines cross-regulate one another. For example, expression of Pim-1, Pim-2 and Socs-1 are all induced by IFNγ (4, 16), and the interplay between Pim kinases and Socs-1 appears to be important for IFNγ-induced inhibition of IL-4 signaling (24-27). In summary, the interaction between Socs-1 and the Pim kinases appears to be important in regulating various signaling pathways in cells of both hematopoietic and non-hematopoietic origin.

References

1. Leonard, W. J. & O'Shea, J. J. (1998) Annu Rev Immunol 16, 293-322.

2. Endo, T. A., Masuhara, M., Yokouchi, M., Suzuki, R., Sakamoto, H., Mitsui, K., Matsumoto, A., Tanimura, S., Ohtsubo, M., Misawa, H., Miyazaki, T., Leonor, N., Taniguchi, T., Fujita, T., Kanakura, Y., Komiya, S. & Yoshimura, A. (1997) Nature 387, 921-4.

3. Naka, T., Narazaki, M., Hirata, M., Matsumoto, T., Minamoto, S., Aono, A., Nishimoto, N., Kajita, T., Taga, T., Yoshizaki, K., Akira, S. & Kishimoto, T. (1997) Nature 387, 924-9.

4. Starr, R., Willson, T. A., Viney, E. M., Murray, L. J., Rayner, J. R., Jenkins, B. J., Gonda, T. J., Alexander, W. S., Metcalf, D., Nicola, N. A. & Hilton, D. J. (1997) Nature 387, 917-21.

5. Chen, X. P., Losman, J. A. & Rothman, P. (2000) Immunity 13, 287-90.

6. Hilton, D. J., Richardson, R. T., Alexander, W. S., Viney, E. M., Willson, T. A., Sprigg, N. S., Starr, R., Nicholson, S. E., Metcalf, D. & Nicola, N. A. (1998) Proc Natl Acad Sci USA 95, 114-9.

7. Nicholson, S. E., Willson, T. A., Parley, A., Starr, R., Zhang, J. G., Baca, M., Alexander, W. S., Metcalf, D., Hilton, D. J. & Nicola, N. A. (1999) Embo J 18, 375-85.

8. Yasukawa, H., Misawa, H., Sakamoto, H., Masuhara, M., Sasaki, A., Wakioka, T., Ohtsuka, S., Imaizumi, T., Matsuda, T., Ihle, J. N. & Yoshimura, A. (1999) Embo J 18, 1309-20.

9. Narazaki, M., Fujimoto, M., Matsumoto, T., Morita, Y., Saito, H., Kajita, T., Yoshizaki, K., Naka, T. & Kishimoto, T. (1998) Proc Natl Acad Sci USA 95, 13130-4.

10. Zhang, J. G., et al. (1999) Proc Natl Acad Sci USA 96, 2071-6.

11. Kamura, T., Sato, S., Haque, D., Liu, L., Kaelin, W. G., Jr., Conaway, R. C. & Conaway, J. W. (1998) Genes Dev 12, 3872-81.

12. Cuypers, H. T., Selten, G., Quint, W., Zijlstra, M., Maandag, E. R., Boelens, W., van Wezenbeek, P., Melief, C. & Berns, A. (1984) Cell 37, 141-50.

13. Lilly, M., Le, T., Holland, P. & Hendrickson, S. L. (1992) Oncogene 7, 727-32.

14. Dautry, F., Weil, D., Yu, J. & Dautry-Varsat, A. (1988) J Biol Chem 263, 17615-20.

15. Domen, J., van der Lugt, N. M., Laird, P. W., Saris, C. J., Clarke, A. R., Hooper, M. L. & Berns, A. (1993) Blood 82, 1445-52.

16. Yip-Schneider, M. T., Horie, M. & Broxmeyer, H. E. (1995) Blood 85, 3494-502.

17. Jacobs, H., Krimpenfort, P., Haks, M., Alien, J., Blom, B., Demolliere, C., Kruisbeek, A., Spits, H. & Berns, A. (1999) J Exp Med 190, 1059-68.

18. Durfee, T., Becherer, K., Chen, P. L., Yeh, S. H., Yang, Y., Kilburn, A. E., Lee, W. H. & Elledge, S. J. (1993) Genes Dev 7, 555-69.

19. Losman, J. A., Chen, X. P., Hilton, D. & Rothman, P. (1999) J Immunol 162, 3770-4.

20. Naka, T., Matsumoto, T., Narazaki, M., Fujimoto, M., Morita, Y., Ohsawa, Y., Saito, H., Nagasawa, T., Uchiyama, Y. & Kishimoto, T. (1998) Proc Natl Acad Sci USA 95, 15577-82.

21. Alexander, W. S., Starr, R., Fenner, J. E., Scott, C. L., Handman, E., Sprigg, N. S., Corbin, J. E., Cornish, A. L., Darwiche, R., Owczarek, C. M., Kay, T. W., Nicola, N. A., Hertzog, P. J., Metcalf, D. & Hilton, D. J. (1999) Cell 98, 597-608.

22. Marine, J. C., Topham, D. J., McKay, C., Wang, D., Parganas, E., Stravopodis, D., Yoshimura, A. & Ihle, J. N. (1999) Cell 98, 609-16.

23. Schmidt, T., Karsunky, H., Rodel, B., Zevnik, B., Elsasser, H. P. & Moroy, T. (1998) Embo J 17, 5349-59.

24. te Velde, A. A., Rousset, F., Peronne, C., De Vries, J. E. & Figdor, C. G. (1990) J Immunol 144, 3052-9.

25. Lee, C. E., Yoon, S. R. & Pyun, K. H. (1993) Mol Immunol 30, 301-7.

26. Venkataraman, C., Leung, S., Salvekar, A., Mano, H. & Schindler, U. (1999) J Immunol 162, 4053-61.

27. Dickensheets, H. L., Venkataraman, C., Schindler, U. & Donnelly, R. P. (1999) Proc Natl Acad Sci USA 96, 10800-5.

28. Danial, N. N., Pernis, A. & Rothman, P. B. (1995) Science 269, 1875-7.

29. Danial, N. N., Losman, J. A., Lu, T., Yip, N., Krishnan, K., Krolewski, J., Goff, S. P., Wang, J. Y. & Rothman, P. B. (1998) Mol Cell Biol 18, 6795-804.

30. van der Lugt, N. M., Domen, J., Verhoeven, E., Linders, K., van der Gulden, H., Alien, J. & Berns, A. (1995) Embo J 14, 2536-94.

31. Lu, B., Reichel, M., Fisher, D. A., Smith, J. F. & Rothman, P. (1997) J Immunol 159, 1255-64.

What is claimed is:

1. A method for treating an allergic response in a subject which comprises administering to the subject a therapeutically effective amount of an agent which increases the amount and/or the activity of a Pim kinase.

2. A method for treating an allergic response in a subject which comprises administering to the subject a therapeutically effective amount of an agent which increases the phosphorylation of a Socs-1 protein by a Pim kinase.

3. A method for treating asthma in a subject which comprises administering to the subject a therapeutically effective amount of an agent which increases the amount and/or the activity of a Pim kinase.

4. A method for treating asthma in a subject which comprises administering to the subject a therapeutically effective amount of an agent which increases the phosphorylation of a Socs-1 protein by a Pim kinase.

5. A method for inhibiting the onset of rejection of a transplanted organ, tissue, or cell in a transplant recipient which comprises administering to the transplant recipient a prophylactically effective amount of an agent which increases the amount and/or the activity of a Pim kinase.

6. A method for inhibiting the onset of rejection of a transplanted organ, tissue, or cell in a transplant recipient which comprises administering to the transplant recipient a prophylactically effective amount of an agent which increases the phosphorylation of a Socs-1 protein by a Pim kinase.

7. The method of any of claims 1-6, wherein the agent is a small molecule.

8. The method of any of claims 1-6, wherein the agent is a polypeptide.

9. The method of any of claims 1-6, wherein the agent is a nucleic acid.

10. The method of claim 1 or 2, wherein the allergic response is characterized by inflammation.

11. The method of claim 1 or 2, wherein the allergic response is characterized by hives, swelling, pain, itching, or redness of skin in the subject.

12. The method of claim 5 or 6, wherein the transplanted organ is a kidney, a heart, an eye, a lung, a stomach, an intestine, an ovary, a pancreas, or at least a portion of liver.

13. The method of claim 5 or 6, wherein the transplanted tissue is skin, brain, muscle, bone, cartilage, or lung.

14. The method of claim 5 or 6, wherein the transplanted cell is an islet cell, a bone marrow cell, a blood cell, a bone cell, a cartilage cell, a stem cell, or a plasma cell.

15. A method for determining whether an agent increases the phosphorylation of a Socs-1 protein by a Pim kinase, which comprises:
(a) contacting the Socs-1 protein, the Pim kinase, and the agent under conditions which would permit phosphorylation of the Socs-1 protein by the Pim kinase in the absence of the agent;
(b) measuring the level of phosphorylation of the Socs-1 protein resulting from step (a); and
(c) comparing that level with the level of phosphorylation of the Socs-1 protein in the absence of the agent,
a higher level of phosphorylation in the presence of the agent indicating that the agent increases the phosphorylation of the Socs-1 protein by the Pim kinase.
